(19) United States
(12) Patent Application Publication
(10) Pub. No.: US 2006/0089550 A1
(43) Pub. Date: Apr. 27, 2006

(54) Disposable container for use with an open magnetic resonance scanner

(76) Inventors: Richard Ian Kitney, London (GB); Patrick Gross, London (GB)

Correspondence Address: MORGAN & FINNEGAN, L.L.P., 3 WORLD FINANCIAL CENTER, NEW YORK, NY, 10281-2101, US

(21) Appl. No.: 10/512,404

(22) PCT Filed: May 30, 2003
(86) PCT No.: PCT/GB03/02372

§ 371 Date: Sep. 8, 2005

(30) Foreign Application Priority Data
May 30, 2002 (GB) 0212581.3

(51) Int. Cl. A61B 5/05

(52) U.S. Cl. 600/410

(57) ABSTRACT

A disposable container (100) which can be used to examine excised material during a surgical procedure is adapted for use with an open magnetic resonance scanner. The container (100) has a receive coil (200), provided in or on the body of the container (100), and can be connected (directly or indirectly) to an input of the scanner. During a surgical procedure which is monitored in known manner by magnetic resonance imaging, using the scanner, excised material such as a tumour can be placed in the container and imaged independently to check for example that the tumour and a margin of healthy tissue has been removed. Conveniently, the container can be placed in the field of the open magnetic resonance scanner for imaging, using its receive coil. The container can also be used in magnetic resonance spectroscopy and tumour classification.

The present invention relates to a medical analysis device and to methods for fabricating and/or using such a device. It finds particular application in magnetic resonance spectroscopy or imaging, for instance for analysis associated with surgery.

Magnetic resonance imaging (“MRI”) for medical diagnosis is well known. Typically, the entire patient or at least that part of the patient to be studied is placed with the main magnetic field of an MRI scanner's magnet. This very strong, homogeneous and static main magnetic field (e.g. 0.5 T) causes the nuclear spins within the patient to align themselves with and against the magnetic field, thereby creating a net magnetic moment in each volume. A transmit coil, typically surrounding the patient, is then used to transmit an excitation pulse which flips the magnetic moment away from its equilibrium position. As the magnetisation decays back to its equilibrium state the spins gyrate around the equilibrium axis and emit signal at the frequency of gyration. This radio frequency signal may be picked up by a receive coil. The frequency of gyration is given by the local magnetic field strength experienced by each spin. This local magnetic field strength is dynamically altered using the scanner's gradients. These superimposed gradients, one in each of the x, y and z directions, typically work at audio frequencies and provide the frequency and phase encoding needed for image reconstruction.

In minimally-invasive MRI, a separate, small, receiving coil or antenna is used to receive the signal, instead of or in addition to the radio-frequency coils of the MRI apparatus itself. Such a coil may be placed either adjacent to the patient's skin or may be inserted by means of a probe into a patient's body cavity, for example into the colon.

MRI is increasingly used during surgical procedures, particularly where tumours or other soft tissue needs to be removed. The area of interest is imaged by the MRI scanner, as surgery proceeds, with both the tumour and the cutting instrument being visible on screen. It is important to remove tumours and their margins without cutting into the affected tissue. Current practice for tumour removal is to mark the tumour boundaries under image guidance (e.g. x-ray, MRI) using markers such as titanium wires, and then to cut around these markers. The result is a lump of tumour, which should have a margin (shell) of healthy tissue surrounding it. It is critical that no off-shoots are missed. Usually this is confirmed by checking that all the markers have been removed and that the tumour is thus completely resected. Furthermore, the operative site is scanned to identify abnormal tissue left and the removed (resected) tissue is placed into a tumour container or resection jar and sent off to a laboratory for further analysis. At the laboratory, x-rays may be taken and frozen sections made.

There are a number of advantages for using MRI for soft tissue imaging. Firstly, the technique does not use ionising radiation. Secondly, no harmful contrast agents are needed. The technique is truly three-dimensional. However, the main advantage is the unrivalled soft-tissue differentiation. MRI may show tumours and tumour margins indistinguishable from healthy tissue in other imaging modality, the naked eye or even to palpation.

In practice, there are a number of problems however. In particular, x-ray analysis of the tumour is not particularly efficient since x-rays do not provide high quality images of soft tissue. Although it would be possible to provide improved images by placing the tumour into another MRI scanner at the laboratory, that would be expensive and would mean either purchasing another scanner to do the analysis or, alternatively, interrupting the routine of a scanner that would normally be used for scanning patients. Because of the time currently taken to analyse the excised tumour, if any problem is found (for example if only part of the tumour has been excised) the patient typically needs to undergo a further surgical operation. That is clearly undesirable.

In a related use of magnetic resonance, there is also increasing interest in spectroscopy of tumours for tumour classification.

According to a first aspect of the present invention, there is provided a container for containing material to be analysed using magnetic resonance, the container including a receive coil for use in analysing material contained in the container.

Such a container can be placed within the magnetic field of a magnetic resonance scanner for use during surgery and material which has been removed from a patient can be placed in the container for imaging, during or immediately after a surgical operation, and preferably while it is still available to resume surgery on the patient.

Thus, using an embodiment of the present invention, when a tumour is removed it can be placed into the container, within the magnetic field of the MR scanner being used during surgery, allowing the tumour to be imaged there and then, in detail. There are several advantages of such an arrangement. Immediately after the surgical operation has been completed, the surgeon can check whether a tumour has been excised cleanly, with a sufficient margin of healthy material around it. This can be done in the operating theatre, while the patient is still in the scanner and at very little additional cost.

The coil included in the container may also be adapted to act as a transmit coil for use in analysing material contained within the container. In such an arrangement, it may not be necessary that the container be placed in the transmit coil of the MR scanner being used during surgery since the container coil itself, acting in transmit mode might be used to create the necessary excitation pulse. Typically, the container will still be positioned within the main magnetic field and the gradients produced by the MR scanner, since the coil may provide an excitation pulse only without simultaneously being used as a receive coil. However, it would be possible for the coil to be used for both transmit and receive.

Preferably, the container is sealable; it may also have access points (e.g. rubber membranes) for biopsies.

Once the surgeon has finished, the container may be sent to a laboratory or other facility for x-rays and/or other analyses to be carried out in the normal way.

Preferably, the container is provided with a connector for connecting the receive coil, directly or indirectly, to an input of a magnetic resonance scanner. In this way, the container can be manufactured as a throw-away item, including just the container with its receive coil and the connector.

In more detail, receive coils for magnetic resonance may in use comprise an inductive part which is connected to circuitry such as matching and decoupling circuits. Preferably, only the inductive part is provided for the container. The more expensive circuitry can be provided as part of the input to a scanner, or as an adaptor for connecting the receive coil to a scanner. The use of an adaptor may be preferred since it supports the use of a single container design with multiple different scanners and/or field strengths. The adaptor can be scanner-specific while the container is a standard item, without there being any need to manufacture a different container for each model of scanner. Containers may however be manufactured in a variety of shapes and sizes, according to application.

Thus in embodiments of the present invention the design can be flexible. It can use standardised scanner independent parts as throw away items. Expensive and specialised parts can all be multi-use.

Preferably, the receive coil is constructed as a volume coil so that material to be imaged can be placed inside the coil. This can provide improved resolution images. Such an arrangement might be particularly suitable for use with an “open” scanner in which patient scans can be carried out during surgical procedures, the surgeon often standing at least partially within the field of the scanner. Open scanners bring huge advantages in guiding the surgeons but current open MRI scanners have low to medium field strengths.

The receive coil could be provided in different ways in relation to the container, and could indeed provide at least part of the containment. It could be formed as part of the container body, or it may be added after the container itself has been manufactured. For example, the coil could be created by sputtering techniques, spraying, screen printing, painting etc. To avoid interfering with the scanner's magnetic field, the container, the receive coil and the connector for connecting the receive coil may be made entirely of non-ferromagnetic material.

According to a second aspect of the present invention, there is provided a scanner for use in analysis by magnetic resonance, the scanner having detachably connected thereto a container comprising at least in part a receive coil for use in analysing material contained in the container.

The scanner may be an open scanner, provided with at least one transmit coil and at least one receive coil which can be arranged for use in imaging the area of a surgical procedure while it is being carried out. The container may be arranged so that it can be disposed in said area to enable analysis of material in the container by use of said at least one transmit coil, together with the receive coil comprised by the container. Alternatively or additionally, the receive coil of the container may also be adapted to function as a transmit coil for use in analysis of material in the container.

An adaptor may be provided between the scanner and the container for adapting the detachable connection to meet requirements of the scanner and of the receive coil of the container in said use in analysing material contained in the container.

According to a third aspect of the present invention, there is provided a method of analysing material by use of magnetic resonance, the method comprising the steps of:
i) generating a main magnetic field for use in analysing a body of material positioned in the field;
ii) removing analysed material from said body of material;
iii) placing the removed material in a container which includes a receive coil, and placing the container in the magnetic field;
iv) applying an excitation pulse to the removed material; and
v) using the receive coil of the container in analysing the removed material.
   Thus a method of analysis is provided in which some material is analysed twice, using MRI or MRS, once while in a body of material and once again after being removed therefrom. The second analysis may be carried out next to the body or at some other, second locality remote from a first locality where the body is situated.

The receive coil of the container might also be adapted to act as a transmit coil, in which case the excitation pulse used for analysis of the material while still in the body of material may be different from the excitation pulse used for analysis of the material after being removed therefrom. However, alternatively, the same excitation pulse could be used for analysis of the material while still in the body of material and after being removed therefrom. The main magnetic field is preferably provided by the main magnet of an MRI scanner, with gradients being supplied by the scanner's gradient coils.

According to a fourth aspect of the present invention, there is provided a method of analysing a sample material by use of magnetic resonance, the method comprising the steps of:
i) placing the sample material in a sample container having a receive coil;
ii) using a transmit coil external to the container to apply an excitation pulse to the sample material; and
iii) using the receive coil to analyse the sample material by use of magnetic resonance in response to the excitation pulse.

In this fourth aspect, embodiments of the present invention can provide a method of analysing material by placing the material in a container with a receive coil and putting the container in a magnetic field generated independently and externally (e.g. by a MRI scanner). In such a method, there may be at least two receive coils, a first receive coil for use with the scanner in the normal way, in the absence of the container, and a second receive coil being the receive coil of the container.

In the above, reference is made to “analysis”, “analysing” and the like. These are not intended to have any restricted special meaning and should be taken to encompass any operation that might be carried out using magnetic resonance such as imaging, spectroscopy, or characterisation for example, as well as any other suitable form of analysis such as microscopy or biochemical analysis.

Embodiments of the present invention will now be described, by way of example only, with reference to the accompanying drawings in which:

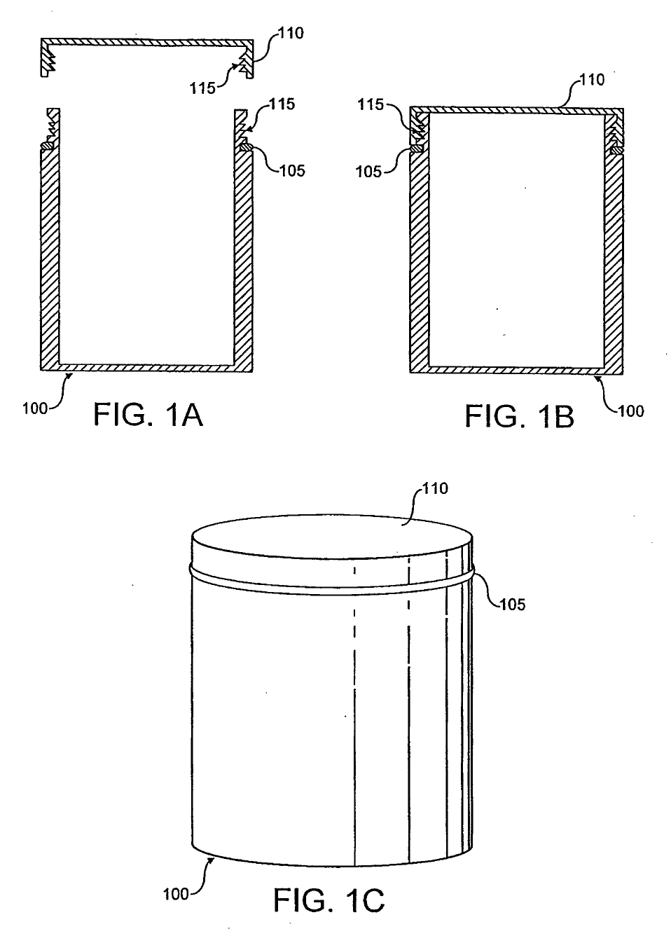

FIG. 1 shows a resection jar comprising a container according to an embodiment of the present invention;

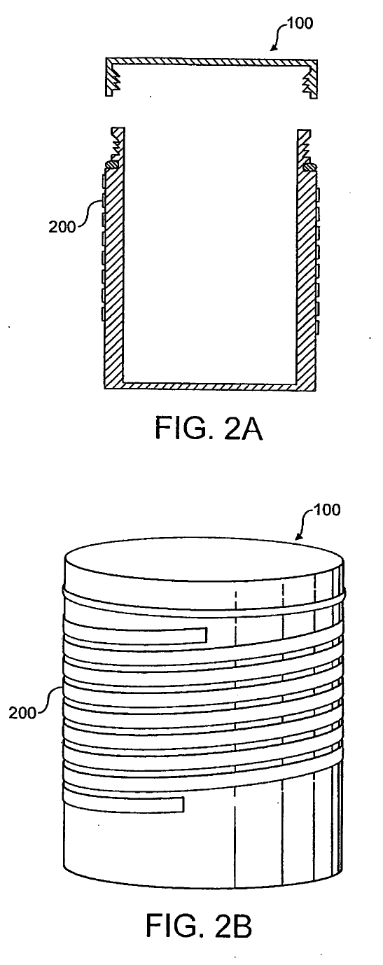

FIG. 2 shows a receive coil for use with the resection jar of FIG. 1;

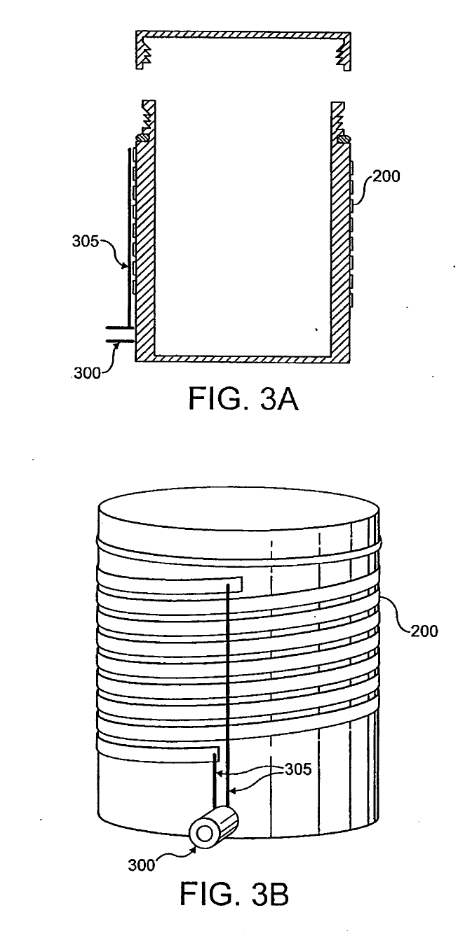

FIG. 3 shows a connector for connecting the receive coil of FIG. 2 to a scanner input;

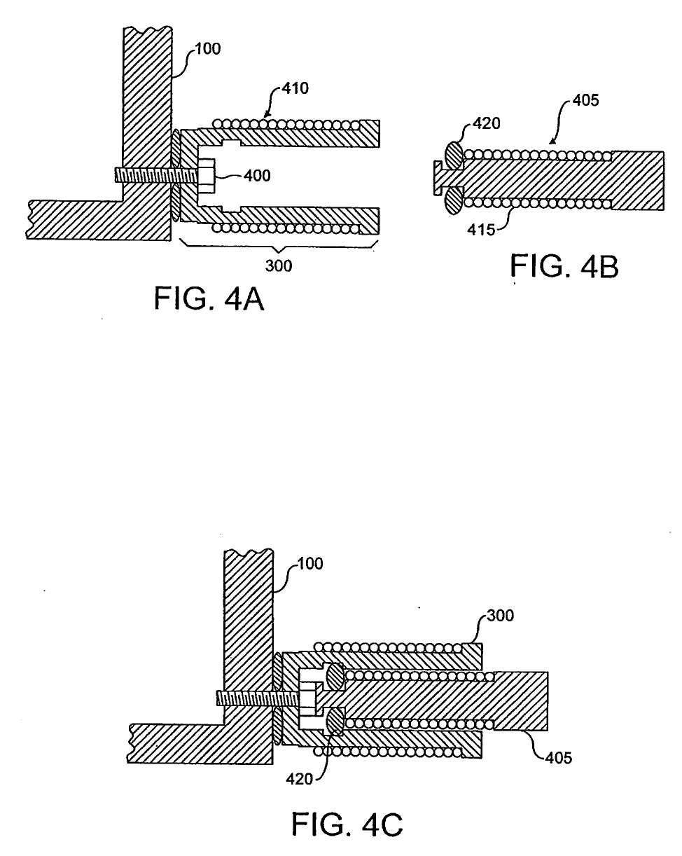

FIG. 4 shows details of an inductively coupled example of the connector of FIG. 3;

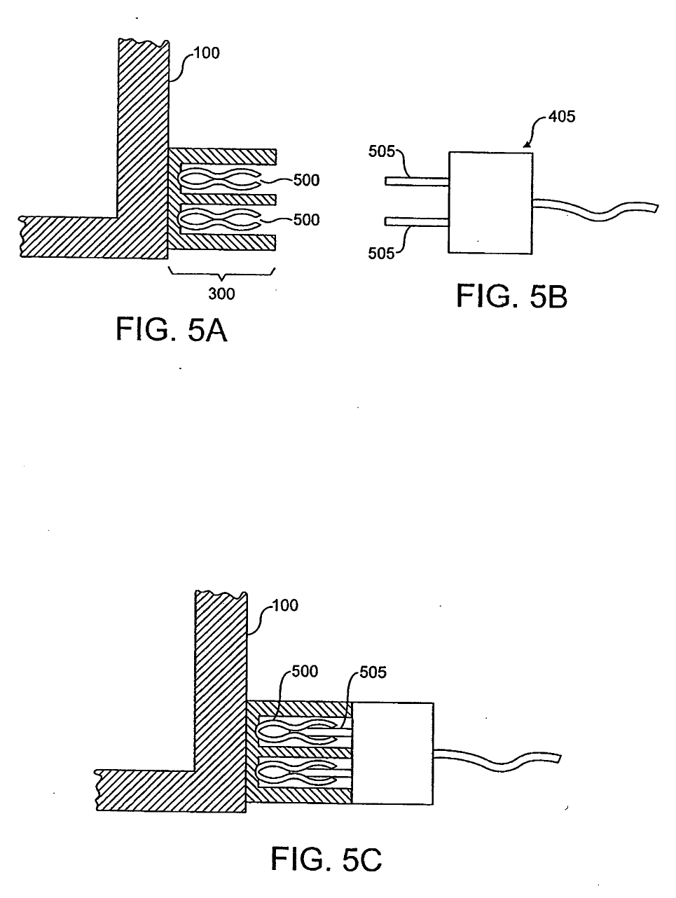

FIG. 5 shows details of a directly coupled example of the connector of FIG. 3;

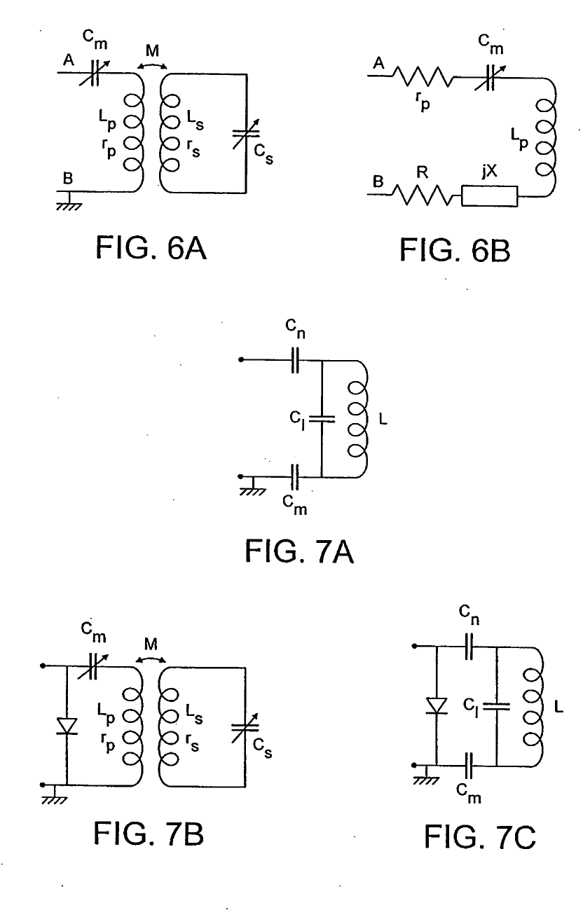

FIG. 6 shows a circuit for use with the inductively coupled connector of FIG. 4;

FIG. 7 shows further coupling circuits; and

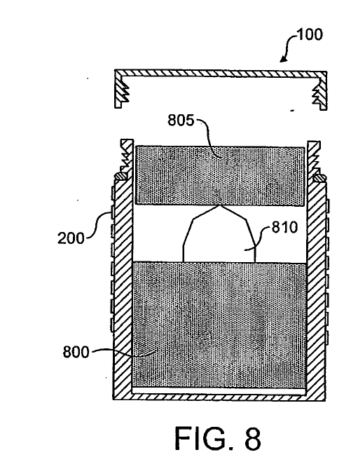

FIG. 8 shows a cross section of the resection jar of FIG. 1 with a sample positioned in the jar for imaging.

Referring to FIG. 1, the resection jar can be a simple container 100 made of clear material with an o-ring seal 105 and a threaded lid 110. In FIG. 1(a), the container 100 is shown open and in cross section, exposing the thread 115 to attach the lid 110 to the body of the container 100. In FIG. 1(b), the container 100 is shown closed and in cross section. In FIG. 1(c), the container 100 is shown closed and substantially in side view, in three dimensions.

The material of the container 100 is preferably transparent so that the position of a sample in it can be seen. Further, as mentioned above, the container and all its parts should be made entirely of non-ferromagnetic material. For example, the lid and body might be made of perspex or glass. The container may include access points, such as rubber-membrane-covered apertures (not shown) via which biopsies may be taken.

Referring to FIG. 2, a solenoid coil 200 is wound onto the outside of the container 100 using for example adhesive copper tape. Solenoid coils are simple and give a good homogeneous magnetic field. The coil 200 could be used as a receive-only antenna, or as a transmit/receive antenna. As the scanner's main magnetic field needs to be perpendicular to the solenoid coil's axis, the configuration is well suited for scanners with horizontal magnetic fields and this includes most scanners. For scanners with vertical fields, the container 100 could either be turned onto its side, or a different configuration could be used.

Referring to FIG. 3, it is necessary to connect the ends of the coil 200 to a connector 300 for connecting the container to a scanner for use. This can be done using enamelled copper wire 305 which is stripped at the contact points and soldered to the ends of the coil 200. It is then attached to the connector 300.

Referring to FIG. 4, depending on the material of the container, the connector 300 could be glued to or screwed into the container 100. FIG. 4 shows a version in which a screw 400 is used. It might be necessary to use several screws. The ruggedness of the mounting would need to be adapted to suit the proposed use of the container. At the end of assembly, the entire container 100 is coated with an epoxy resin or other insulating, sealing and hardening varnish.

Referring to FIGS. 4 and 5, at least two different options exist for the design of the connector 300. The first option is to use inductive coupling, as shown in FIG. 4. The second option is to use a direct connection, as shown in FIG. 5. A third option would be to connect the coil to the scanner by means of a wireless connection using a transmitter and receiver, ensuring that the transmitted signal does not interfere with the operation of the scanner, and vice versa. An example of such a wireless link is a connection by means of an infrared optical signal.

Taking the first option, the connector 300 shown in FIG. 4(a) provides the socket of a plug and socket arrangement. The plug component 405 is shown in FIG. 4(b) and the two parts are shown coupled together and held by an O-ring 420 in FIG. 4(c). The socket 300 and the plug component 405 each contain a coaxial solenoid 410, 415 and their mutual inductance is used to transfer energy from the solenoid coil 200 towards the scanner. To put it another way, the solenoid 410 adds to or forms part of the coil inductance.

Taking the second option, as shown in FIG. 5(a), the connector 300 again provides the socket of a plug and socket arrangement but this time it is equipped with two pairs of resilient conductive blades 500. A plug component 405 for this form of the connector 300 is shown in FIG. 5(b) and this is equipped with two conductive prongs 505 which can be pushed between the conductive blades 500 to complete the coupling, as shown in FIG. 5(c). Each conductive prong 505 couples to a wire 305 such as is shown in FIG. 3.

Both types of coupling are known per se, inductive and direct. FIGS. 6 and 7 show the circuitry suggested by each respectively. Such circuitry might be provided at an input to a scanner, or (preferably) as part of an adaptor for connecting the container 100 to a scanner. The adaptor will typically be proximal to the container.

The circuits of FIGS. 6 and 7 are disclosed in “An inductively coupled, series-tuned NMR probe”, M. Decords, P. Blondet, H. Reutenauer, J. P. Albrand Journal of Magnetic Resonance 65, 100-109 (1985).

Referring to FIG. 6, for the inductively coupled circuit the container 100 will have to have a mounted capacitor Cₛ. Ideally it should be chosen to match a given field strength. However, it is also possible to use the same container for a range of field strengths, making more extensive provisions on the socket side. FIGS. 6(a) and 6(b) show equivalent circuits. At the socket side, we have control over Lₚ, rₚ, Cₘ and M. Typically, rₚ should be roughly zero, and M should be high. M, Lₚ and Cₘ may be adjusted to achieve matching.

Referring to FIG. 7a, from the circuit point of view, the direct contact arrangement shown in FIG. 5 is simpler. The container 100 need not have any components mounted other than the coil 200 and it may be used for all field strengths.

A decoupling scheme may need to be added if the coil 200 on the container 100 is to be used as receive only. This may be achieved using a DC bias on the coaxial cable to switch a diode at the input terminals of the matching section, as is shown in FIGS. 7(b) and 7(c).

Referring to FIG. 8, material 800, 805 such as foam, preferably non-water-absorbing, may be inserted in the container 100 to ensure correct positioning of the sample 810 in the container 100. Once the sample 810 is positioned inside the container, the container can be sealed and/or labelled inside the operating theatre, facilitating transport of the sample to a second site, such as a laboratory, for storage or further analysis. This is particularly relevant in case that the second site is located in a different part of the hospital where the operating theatre is located, or in an entirely different building.

Various design features may be preferred for use of the container 100. For instance, it may be preferred that the base of the container is made anti-slip and a label area might be provided on the outside. The entire container is preferably disposable and intended for single-use only. Alternatively, the container may be made of suitable materials to allow sterilisation and re-use.

In the above, attention is given to imaging of material, particularly to support surgical procedures. However, there may be other applications to which embodiments of the invention would be relevant. An example of one of these is magnetic resonance spectroscopy of tumours for tumour classification.

1. A container for containing material to be analysed using magnetic resonance, the container including a receive coil for use in analysing material contained in the container and a connector for detachably connecting the receive coil, directly or via an adaptor, to an input of a magnetic resonance scanner.

2. A container according to claim 1, wherein the connector provides an inductive coupling to the receive coil.

3. A container according to claim 1, wherein the connector provides a direct electrical contact to the receive coil.

4. A container according to claim 1, wherein the connector includes a transmitter for establishing a wireless link between the receive coil and the input of the magnetic resonance scanner.

5. A container according to claim 1 wherein the receive coil is also adapted for use as a transmit coil for use in analyzing material contained in the container.

6. A container according to claim 1 wherein the container is sealable.

7. A container according to claim 1 wherein the receive coil is constructed as a volume coil such that material to be analyzed can be placed inside the coil.

8. A container according to claim 1 wherein the container is made of non-ferromagnetic material such that material contained in the container can be analyzed by use of an excitation pulse generated by use of at least one transmit coil external to the container.

9. A scanner for use in analysis by magnetic resonance, the scanner having detachably connected thereto a container including a receive coil for use in analysing material contained in the container.

10. A scanner according to claim 9, said scanner being an open scanner, provided with at least one transmit coil and at least one receive coil arranged for use in imaging a three-dimensional space in which a surgical procedure can be at least partially carried out.

11. A scanner according to claim 10 wherein the container is disposed in said space to enable analysis of material contained in the container by use of said at least one transmit coil, together with the receive coil of the container.

12. A scanner according to claim 9 wherein the receive coil of the container is adapted to function additionally as a transmit coil for use in analysis of material contained in the container.

13. A scanner according to claim 9, the scanner being provided with an adaptor between the scanner and the container, for adapting the detachable connection to meet requirements of the scanner and of the receive coil of the container in said use in analyzing material contained in the container.

14. A method of analysing material by use of magnetic resonance, the method comprising:
i) generating a main magnetic field for use in analysing a body of material positioned in the field;
ii) removing analysed material from said body of material;
iii) placing the removed material in a container which includes a receive coil; and
iv) placing the container in the magnetic field;
v) applying an excitation pulse to the removed material; and
vi) using the receive coil of the container in analysing the removed material.

15. A method according to claim 14 wherein the body is at least initially situated in a first locality; the method further comprising
vii) sealing the container at the first locality.

16. A method according to claim 14 wherein the body is at least initially situated in a first locality; the method further comprising
viii) labelling the container at the first locality.

17. A method according to claim 14 wherein the body is at least initially situated in a first locality; the method further comprising
ix) transporting the container to a second locality.

18. A method according to claim 14 wherein the body is at least initially situated in a first locality; the method further comprising
x) further analysing the removed material at a second locality.

19. A method according to claim 14 wherein the receive coil of the container is adapted also to act as a transmit coil, for generating the excitation pulse.

20. A method according to claim 14 wherein the excitation pulse is generated by a transmit coil external to the container.

21. A method according to claim 14 wherein the body is a patient and the first locality is an operating theatre.

22. A method of analysing body tissue by use of magnetic resonance, the method comprising:
i) generating a main magnetic field for use in analysing a body tissue of a patient positioned in the field;
ii) resecting the analysed body tissue from said patient;
iii) placing the resected body tissue in a container which includes a receive coil; and
iv) placing the container in the magnetic field;
v) applying an excitation pulse to the resected body tissue; and
vi) using the receive coil of the container in analysing the resected body tissue.

23. A method according to claim 22, wherein the patient is situated in an operating thratre.

24. A method according to claim 23 further comprising
vii) sealing the container inside the operating thratre.

25. A method according to claim 23 further comprising
viii) labelling the container inside the operating thratre.

26. A method according to claim 23 further comprising
ix) further analysing the resected body tissue outside the operating thratre.

27. A method according to claim 22 wherein the receive coil of the container is adapted also to act as a transmit coil, for generating the excitation pulse.

28. A method according to claim 22 wherein the excitation pulse is generated by a transmit coil external to the container.

29. A method according to claim 22, wherein the body tissue comprises tumour tissue.

30. A method of analysing a sample material at a first locality by use of magnetic resonance, the method comprising the steps of:
i) placing the sample material in a sample container having a receive coil;
ii) using a transmit coil external to the container to apply an excitation pulse to the sample material; and
iii) using the receive coil to analyse the sample material by use of magnetic resonance in response to the excitation pulse.

31. A method as claimed in claim 30 further comprising iv) sealing the container at the first locality.

32. A method as claimed in claim 30 further comprising:
v) labelling the container at the first locality.

33. A method as claimed in claim 30 further comprising:
vi) transporting the container to a second locality.

34. A method as claimed in claim 30 further comprising:
vii) further analyzing the sample material at a second locality.

35. A method as claimed in claim 30 including using apparatus external to the container to provide a main magnetic field within which the sample container is positioned during analysis.

36. A method as claimed in claim 30 including using apparatus external to the container to provide magnetic gradients within which the sample container is positioned during analysis.

37. A method as claimed in claim 30, wherein the sample material comprises body tissue.

38. A method as claimed in claim 30, wherein the sample material comprises tumour tissue.

39. A method as claimed in claim 30, wherein the first locality is an operating theatre.

40. A method as claimed in claim 33, wherein the second locality is a laboratory.
